(19) United States
(12) Patent Application Publication
(10) Pub. No.: US 2013/0178448 A1
(43) Pub. Date: Jul. 11, 2013

(54) Treatment of Respiratory Disorders

(71) Applicants: Wilson Caparros-Wanderley, London (GB); John Brew, London (GB); Gregory Stoloff, London (GB); Robin Mark Bannister, London (GB)

(72) Inventors: Wilson Caparros-Wanderley, London (GB); John Brew, London (GB); Gregory Stoloff, London (GB); Robin Mark Bannister, London (GB)

(21) Appl. No.: 13/576,758

(22) PCT Filed: Feb. 4, 2011
(86) PCT No.: PCT/GB2011/050189

§ 371 Date: Dec. 14, 2012

(30) Foreign Application Priority Data
Feb. 5, 2010 (GB) 1001912.3
Sep. 22, 2010 (GB) 1015878.0

(51) Int. Cl. A61K 47/46; A61K 47/10; A61K 31/616; A61K 31/192

(52) U.S. Cl. CPC A61K 47/46; A61K 31/192; A61K 47/10; A61K 31/616
USPC 514/165; 514/570

(57) ABSTRACT

This invention relates to the treatment of respiratory disorders, and in particular respiratory disorders and oedema caused by pathogenic infections. In particular, the invention relates to orally administrable pharmaceutical compositions for treating respiratory disorders, and to methods of such treatment. The invention is particularly concerned with the treatment of respiratory disorders that are caused by viral infections, such as with influenza viral strains. The invention also extends to analgesic compositions and methods for treating inflammatory pain manifesting in a variety of diseases, and not only respiratory diseases.

This patent application claims priority pursuant to 35 U.S.C. §365(b) to International Patent Application Serial No. PCT/GB2010/050189 filed Feb. 4, 2011, which claims priority to GB 1015878.0. filed Sep. 22, 2010 and GB 1001912.3, filed Feb. 5, 2010, each of which is hereby incorporated by reference in its entirety.

The present invention relates to the treatment of respiratory disorders, and in particular respiratory disorders and oedema caused by pathogenic infections. In particular, the invention relates to orally administrable pharmaceutical compositions for treating respiratory disorders, and to methods of such treatment. The invention is particularly concerned with the treatment of respiratory disorders that are caused by viral infections, such as with influenza viral strains, including not only existing viruses, but also future, derivative strains of viruses that have mutated from existing viruses, which could give rise to an influenza pandemic. The invention also extends to analgesic compositions and methods for treating inflammatory pain manifesting in a variety of diseases, and not only respiratory diseases.

Respiratory disease is the term used for diseases of the respiratory system, and includes diseases of the upper and lower respiratory tract, such as the lung, pleural cavity, bronchial tubes, trachea, and of the nerves and muscles that are involved with breathing. Respiratory diseases can be mild and self-limiting, such as the common cold, and so often pass without the need for treatment. However, respiratory disease can also be life-threatening, such as bacterial or viral pneumonia, and so extra care and additional treatment can be required for people who are more vulnerable to the effects of microbial infections, such as the very young, the elderly, people with a pre-existing lung condition, and people with a weakened immune system.

Treatment of respiratory disease depends on the particular disease being treated, the severity of the disease and the patient. Vaccination can prevent certain respiratory diseases, as can the use of antibiotics. However, the growth in viral and fungal infections, and the emergence of antimicrobial drug resistance in human bacterial pathogens is an increasing problem worldwide. Moreover, since the introduction of antimicrobials, the emergence of resistance has become increasingly prevalent, particularly for important pathogens, such as E.coli and Staphylococcus spp. As a consequence, effective treatment of such micro-organisms and the control of respiratory diseases is becoming a greater challenge.

The defence against disease is critical for the survival of all animals, and the mechanism employed for this purpose is the animal immune system. The immune system is very complex, and involves two main divisions, (i) innate immunity, and (ii) adaptive immunity. The innate immune system includes the cells and mechanisms that defend the host from infection by invading organisms, in a non-specific manner. Leukocytes, which are involved with the innate system, include inter alia phagocytic cells, such as macrophages, neutrophils and dendritic cells. The innate system is fully functional before a pathogen enters the host.

In contrast, the adaptive system is only initiated after the pathogen has entered the host, at which point it develops a defence specific to that pathogen. The cells of the adaptive immune system are called lymphocytes, the two main categories of which are B cells and T Cells. B cells are involved in the creation of neutralising antibodies that circulate in blood plasma and lymph and form part of the humoral immune response. T cells play a role in both the humoral immune response and in cell-mediated immunity. There are several subsets of activator or effector T cells, including cytotoxic T cells (CD8+) and “helper” T cells (CD4+), of which there are two main types known as Type 1 helper T cells (Th1) and Type 2 helper T cell (Th2).

Th1 cells promote a cell-mediated adaptive immune response, which involves the activation of macrophages and stimulates the release of various cytokines, such as IFNγ, TNF-α and IL-12, in response to an antigen. These cytokines influence the function of other cells in the adaptive and innate immune responses, and result in the destruction of micro-organisms. Generally, Th1 responses are more effective against intracellular pathogens, such as viruses and bacteria present inside host cells. A Th2 response, however, is characterised by the release of IL-4, which results in the activation of B cells to make neutralising antibodies, which lead the humoral immunity. Th2 responses are more effective against extracellular pathogens, such as parasites and toxins located outside host cells. Accordingly, the humoral and cell-mediated responses provide quite different mechanisms against an invading pathogen.

The present invention is concerned with the development of novel compositions for the treatment of disorders of the respiratory tract. The invention is especially concerned with the development of novel therapies for the treatment of a broad range of viral infections, including acute viral infections such as influenza, and in particular, the treatment of respiratory diseases, and oedema, caused thereby.

Despite the requirement for a vaccine for each new virus, most individuals contracting annual flu who have not been vaccinated will nonetheless still have some degree of immune protection against the new virus. This is because the mutations that give rise to the new virus are relatively small, and hence an individual's pre-existing antibody response is still able to provide some degree of protection against the new virus. This pre-existing antibody response has been found to play a significant role in reducing the likelihood of a subject becoming seriously ill or dying as a result of contracting influenza. When an individual's pre-existing antibody response has very little or no capacity to neutralise the new influenza virus strain, the natural cellular immune response that the individual will develop to this new strain can become dominant over the antibody response and develop an uncontrolled inflammatory response leading to severe lung pathology, and even death. This is due to the role played by antibodies in modulating the cellular, and its associated cytokine, immune responses.

Cytokines are produced by many different cell types, some immune and some non-immune cells, and they determine the type and proliferation rate of immune cells engaged in fighting the viral infection. In the absence of a neutralising antibody response, the type and level of cellular immune response, and the cytokine environment created as a result, both change, and are significantly increased. This increased cellular and cytokine response can cause the individual to develop severe impairment of lung function (e.g. pulmonary oedema), leading to death in the most severe cases.

It is known that several cytokines are involved in causing this problem. TNF-α, IL-12 and IFN-γ are three of the most significant cytokines that are believed to be operating. Baumgarth and Kelso (J. Virol., 1996, 70, 4411-4418) reported that neutralisation of the Th1 cytokine, IFN-γ, can lead to a significant reduction in the magnitude of the cellular infiltrate in lung tissue following infection, and suggested that IFN-γ may be involved in the mechanisms that regulate increased leukocyte traffic in the inflamed lung. They also postulated that IFN-γ affects the local cellular response in the respiratory tract, as well as the systemic humoral response to influenza virus infection.

Following on from this study, the inventors of the present invention set out to determine whether the suppression of IFN-γ, and other cytokines, such as TNF-α, might be a possible, and if so, if it could be useful in the treatment of influenza. In their previous experiments, the inventors have demonstrated, using in vitro studies, that certain compounds can be effectively used to decrease the concentrations of IFN-γ and TNF-α in Peripheral Blood Mononuclear Cells (PMBC) that had been stimulated in such a way that they reflected an acute viral infection. The inventors also demonstrated, using in vivo mouse studies, that these same compounds resulted in increased weight and percentage survival rates in influenza-challenged mice. They have therefore postulated that there is a direct link between decreasing concentrations of IFN-γ and TNF-α, and the increased survival rates seen in the mouse studies.

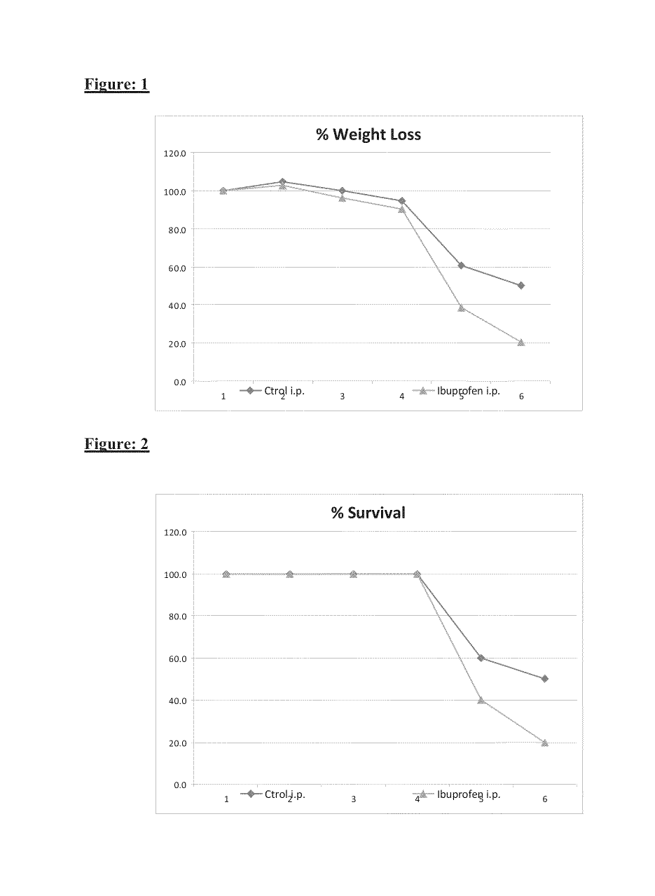

Therefore, based on these previous findings, the inventors then decided to investigate, using in vivo mouse studies, the effects of non-steroidal anti-inflammatory drugs, such as ibuprofen, on mice that had been previously challenged with influenza virus. Ibuprofen was initially administered to the mice intraperitoneally (I.P.) and, as shown in FIGS. 1 and 2, the inventors observed that there did not appear to be any positive effect on either the percentage weight loss or the percentage survival rate in the test mice when compared to the control mice. The inventors therefore reformulated ibuprofen in combination with a lipophilic pharmaceutically acceptable vehicle, which was then orally administered to test mice.

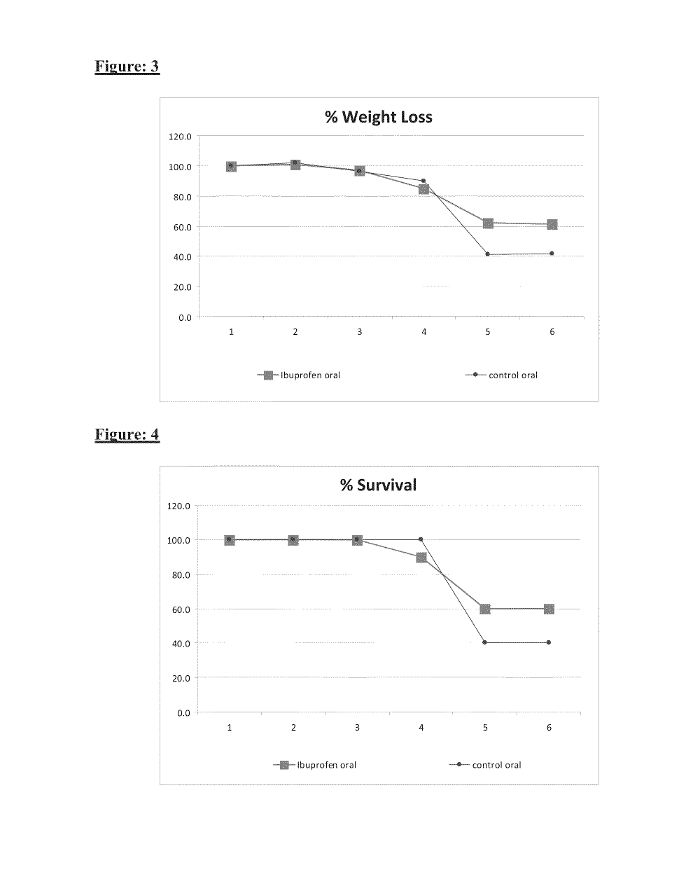

As shown in FIGS. 3 and 4, to their surprise, the inventors observed that, in contrast to intraperitoneally-administered ibuprofen, ibuprofen that had been administered orally in an oily formulation resulted in positive effects on both the percentage weight loss and the percentage survival rate compared to the control mice. The inventors have also shown that the use of the lipophilic vehicle results in the increased bioavailability of ibuprofen in the lung, such that it can impart its effect on the influenza-challenged mice. The inventors believe that their latest findings are not limited to just ibuprofen, and that lipophilic pharmaceutically vehicles may used to improve the oral delivery of any non-steroidal anti-inflammatory drug for use in treating respiratory disorders.

Therefore, in a first aspect of the invention, there is provided a pharmaceutical composition for oral administration, the composition comprising a therapeutically effective amount of a non-steroidal anti-inflammatory drug (NSAID) or a derivative thereof, and a pharmaceutically acceptable vehicle comprising a lipid and an alcohol, wherein the composition is for use in treating a respiratory disorder.

In a second aspect, there is provided a method of preventing, treating and/or ameliorating a respiratory disorder, the method comprising orally administering, to a subject in need of such treatment, a pharmaceutical composition comprising a therapeutically effective amount of a non-steroidal anti-inflammatory drug (NSAID) or a derivative thereof, and a pharmaceutically acceptable vehicle comprising a lipid and an alcohol.

In a third aspect, there is provided a use of a pharmaceutically acceptable vehicle comprising a lipid and an alcohol in an orally administrable pharmaceutical composition, for increasing the bioavailability of a non-steroidal anti-inflammatory drug (NSAID) or a derivative thereof in a subject's lung.

Surprisingly, in contrast to intraperitoneal administration, when ibuprofen is administered orally in a lipophilic formulation, it is shown to be very effective in the treatment of influenza-induced respiratory collapse in mice. Although the inventors do not wish to be bound by any theory, they believe that one explanation for this surprising observation may be due to the lipophilicity of NSAIDs, such as ibuprofen, which, when delivered in an oily formulation having a high lipid content (e.g. at least 30% (w/w) lipid) results in them being rapidly absorbed into the systemic circulation via the lymphatic system. When a drug/lipid formulation is swallowed, the lipids are mixed with bile in the stomach, containing bile salts, and form micelles which are absorbed by the intestine and converted into chylomicrons, which are large lipoprotein particles that consist of triglycerides, phospholipids, cholesterol and proteins, and the NSAID.

The resultant oil/drug chylomicrons may then be absorbed by the proximal gut into the lymphatic system. These chylomicrons, carrying the NSAID, are believed to be transported via the gut lymphatic system to the central venous vasculature, and then rapidly to the heart, which pumps the NSAID-rich venous blood to the lung. As a result, the drug is delivered in high concentrations in oxygenated blood directly to the lung increasing its bioavailability at the treatment site. The inventors believe that lymphatic absorption of the NSAID (e.g. ibuprofen) may be acting as a passive system of distribution of the drug directly to the lung, exposing the lung to high concentrations of the drug; a significant advantage when treating respiratory disorders. The inventors believe that this delivery mechanism does not occur when using intraperitoneal formulations, or standard oral formulations, which contain no, or only low levels of lipid, which are instead absorbed via the hepatic portal vein, with liver-regulated venous absorption, which releases the drug into systemic circulation relatively slowly.

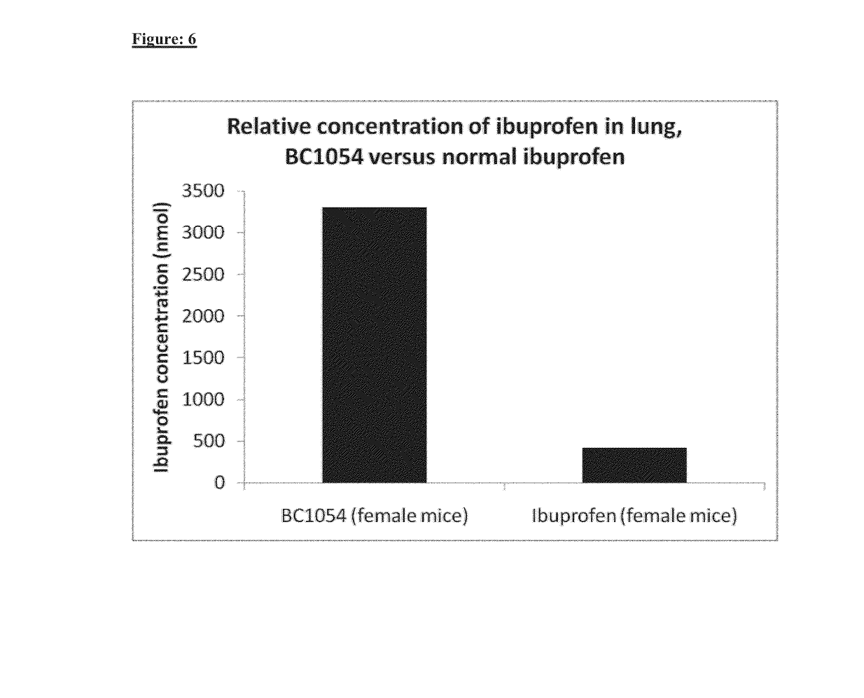

Accordingly, the inventors believe that the high concentration of lipids in the pharmaceutical vehicle used in the composition of the first aspect may be the reason for the effectiveness of the orally-administered ibuprofen in the influenza-induced respiratory collapse assay in mice, as described in the Examples. As convincingly shown in FIG. 6, the concentration of ibuprofen in the lungs of mice administered with the composition of the invention was approximately 8-fold higher than the concentration of ibuprofen in the lungs of the control mice (i.e. animals orally administered with normal ibuprofen). This was totally unexpected, and is a clear demonstration that the composition of the invention results in a surprisingly significant increase in the bioavailability of the NSAID in the lung.

Thus, the vehicle comprising the lipid component may be capable of increasing the concentration of NSAID or derivative thereof in a subject's lung by at least 5%, 10%, 20%, 30%, 50%, 100%, 200%, 300%, 400%, 500%, 600%, 700% or at least 800% compared to that which would be achieved via intraperitoneal administration, or by oral administration using a non-lipid vehicle (as used in Example 2).

The pharmaceutical vehicle may comprise at least about 10%, 20%, 30%, 35%, 40%, 45%, 50%, 55%, 60%, 65%, 70%, 75%, 80%, 85%, 90%, 95% or at least about 99% (w/w) lipid. The vehicle may comprise between about 35% and 99% (w/w) lipid, or between about 45% and 99% (w/w) lipid, or between about 50% and 99% (w/w) lipid, or between about 60% and 98% (w/w) lipid, or between about 70% and 97% (w/w) lipid, or between about 80% and 96% (w/w) lipid, or between about 85% and 95% (w/w) lipid, or between about 85% and 95% (w/w) lipid, or between about 88% and 94% (w/w) lipid, or between about 89% and 93% (w/w) lipid.

The pharmaceutical vehicle may comprise a lipid component selected from a group consisting of: an oil or oil-based liquid; a fat; a fatty acid (e.g. oleic acid, stearic acid or palmitic acid etc.), a fatty acid ester, a fatty alcohol, a glyceride (mono-, di- or tri-glyceride); a phospholipid; a glycol ester; a sucrose ester; a wax; a glycerol oleate derivative; a medium chain triglyceride; or a mixture thereof. A triglyceride is an ester derived from glycerol and three fatty acids, and is the main constituent of vegetable oil and animal fats.

The term “oil” can refer to a fat that is liquid at normal room temperature, and can be used for any substance that does not mix with water, and which has a greasy feel. The term “fat” can refer to a fat that is solid at normal room temperature. The term “lipid” can therefore refer to a liquid or solid fat, as well as to other related substances. A suitable oil, which may be used as the lipid component in the pharmaceutical vehicle, may be a natural oil or a vegetable oil. Examples of suitable natural oils may be selected from a group consisting of linseed oil; soyabean oil; fractionated coconut oil; triacetin; ethyl oleate; a hydrogenated natural oil; or a mixture thereof. Examples of suitable vegetable oils may be selected from a group consisting of rapeseed oil; olive oil; peanut oil; soybean oil; corn oil; safflower oil; arachis oil; sunflower oil; canola oil; walnut oil; almond oil; avocado oil; castor oil; coconut oil; corn oil; cottonseed oil; rice bran oil; sesame oil; and refined palm oil; or a mixture thereof. Each of these oils is commercially available from a number of sources well recognized by those skilled in the art.

The lipid component of the pharmaceutical vehicle may comprise a fatty acid comprising between 8 and 24 carbon atoms, between 10 and 22 carbon atoms, between 14 and 20 atoms, or between 16 and 20 atoms. The lipid may be saturated or unsaturated, for example with one, two, three or more double bonds. The lipid may comprise a fatty acid selected from a group consisting of myristic acid (C 14:0); palmitic acid (C 16:0); palmitoleic acid (C 16:1); stearic acid (C 18:0); oleic acid (C 18:1); linoleic acid (C 18:2); linolenic acid (C 18:3) and arachidic acid (C 20:0); or a mixture thereof. It will be appreciated that the first number provided in the brackets corresponds to the number of carbon atoms in the fatty acid, and that the second number corresponds to the number of double bonds (i.e. unsaturation).

The melting point of the oil is largely determined by the degree of saturation/unsaturation. The melting points of oleic acid (CH₃(CH₂)₇CH═CH(CH₂)₇(COOH), linoleic acid (CH₃(CH₂)₄(CH═CHCH2)₂(CH₂)₆COOH), and of linolenic acid (CH₃CH₂(CH═CHCH₂)₃(CH₂)₆COOH), are about 16° C., −5° C. and −11° C., respectively. Thus, the melting point of the lipid may be between about −20° C. and 20° C., or between about −15° C. and 16° C.

In one embodiment, the lipid component of the pharmaceutical vehicle may comprise olive oil. However, in a preferred embodiment, the lipid may comprise rapeseed oil or linseed oil. Rapeseed oil is derived from Brassica napus, and contains both omega-6 and omega-3 fatty acids in a ratio of about 2:1. Linseed oil, also known as flax seed oil, is a clear to yellowish oil obtained from the dried ripe seeds of the flax plant (Linum usitatissimum, Linaceae). The oil is obtained by cold pressing, sometimes followed by solvent extraction. Linseed oil is a mixture of various triglycerides that differ in terms of their fatty acid constituents. For linseed oil, the constituent fatty acids are of the following types: (i) the saturated acids palmitic acid (about 7%) and stearic acid (3.4-4.6%); (ii) the monounsaturated oleic acid (18.5-22.6%); (iii) the doubly unsaturated linoleic acid (14.2-17%); and (iii) the triply unsaturated omega-3 fatty acid α-linolenic acid (51.9-55.2%). Linseed oil is also rich in omega-6 fatty acid. The structure of a representative triglyceride found in linseed oil may be represented by formula I:

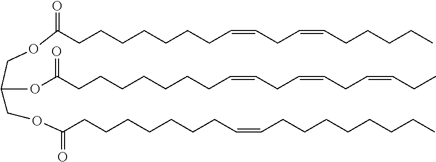 [SMILES: CC/C=C\C/C=C\C/C=C\CCCCCCCC(=O)OC(COC(=O)CCCCCCC/C=C\C/C=C\CCCCC)COC(=O)CCCCCCC/C=C\CCCCCCCC]

Thus, the lipid component of the pharmaceutical vehicle may comprise omega 3 and/or omega 6 fatty acid. Omega-3 fatty acids are a family of unsaturated fatty acids that have in common a final carbon-carbon double bond in the n-3 position, i.e. the third bond from the methyl end of the fatty acid, and can be represented by formula II.

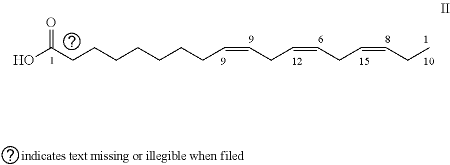 [SMILES: CC/C=C\C/C=C\C/C=C\CCCCCCCC(=O)O]

Omega-6 fatty acids, on the other hand, are a family of unsaturated fatty acids that have in common a final carbon-carbon double bond in the n-6 position, i.e. the sixth bond, counting from the end opposite the carboxyl group, and can be represented by formula

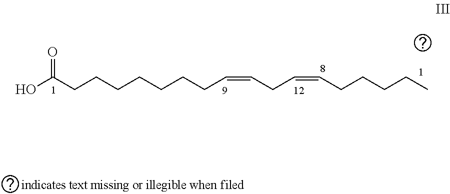 [SMILES: CCCCC/C=C\C/C=C\CCCCCCCC(=O)O]

Omega-3 and omega-6 fatty acids are derivatives of linolenic acid, the main difference being the number and exact position of the double bonds. Accordingly, omega-3 and omega-6 will have substantially the same melting points as linolenic acid.

The vehicle may comprise less than about 90%, 80%, 70%, 65%, 60%, 55%, 50%, 45%, 40%, 35%, 30%, 25%, 20%, 15%, 10%, 5%, or less than about 1% (w/w) alcohol. The vehicle may comprise between about 1% and 90% alcohol (w/w), or between about 1% and 70% (w/w) alcohol, or between about 1% and 60% (w/w) alcohol, or between about 1% and 50% (w/w) alcohol, or between about 2% and 40% (w/w) alcohol, or between about 4% and 30% (w/w) alcohol, or between about 6% and 20% (w/w) alcohol, or between about 8% and 15% (w/w) alcohol. The alcohol may be an aliphatic alcohol. The alcohol may be a C₁₋₂₀ alcohol, a C₁₋₁₅ alcohol, a C₁₋₁₀ alcohol, a C₁₋₅ alcohol, or a C₂₋₄ alcohol. The alcohol may be ethanol, propanol or butanol. In one preferred embodiment, the alcohol is ethanol.

In one embodiment, the vehicle may comprise between approximately 60% and 95% (w/w) oil and between about 5% and 40% (w/w) alcohol. In another embodiment, the vehicle may comprise between approximately 80% and 95% (w/w) lipid and between about 5% and 20% (w/w) alcohol. For example, the vehicle may comprise between approximately 80% and 95% (w/w) olive oil, rapeseed oil or linseed oil, and between approximately 5% and 20% (w/w) ethanol. In another embodiment, the vehicle may comprise between approximately 88% and 92% (w/w) lipid, and between approximately 8% and 12% (w/w) alcohol. For example, the vehicle may comprise between approximately 88% and 92% (w/w) olive oil, rapeseed oil or linseed oil, and between approximately 8% and 12% (w/w) ethanol. In another embodiment, the vehicle may comprise approximately 90% (w/w) lipid, and approximately 10% (w/w) alcohol. For example, the vehicle may comprise approximately 90% (w/w) olive oil, rapeseed oil or linseed oil, and approximately 10% (w/w) ethanol.

The inventors believe that water has a tendency to increase the instability of NSAIDs. Thus, in a preferred embodiment, the vehicle is substantially anhydrous.

Advantageously, the absence of water in embodiments of the vehicle mean that the stability of the NSAID in the composition is not compromised, thereby providing an improved product.

However, in some embodiments, the vehicle may optionally comprise water. The vehicle may comprise less than about 70%, 65%, 60%, 55%, 50%, 45%, 40%, 35%, 30%, 25%, 20%, 15%, 10%, 5%, or less than about 1% (w/w) water. The vehicle may comprise between about 1% and 70% (w/w) water, or between about 1% and 60% (w/w) water, or between about 1% and 50% (w/w) water, or between about 2% and 40% (w/w) water, or between about 4% and 30% (w/w) water, or between about 6% and 20% (w/w) water, or between about 8% and 15% (w/w) water.

The non-steroidal anti-inflammatory drug (NSAID) may be a propionic acid derivative, an acetic acid derivative, an enolic acid derivative, a fenamic acid derivative, or a selective- or non-selective cyclooxygenase (COX) inhibitor. The NSAID may be a profen.

Examples of suitable propionic acid NSAID derivatives may include Ibuprofen; Naproxen; Fenoprofen; Ketoprofen; Flurbiprofen; or Oxaprozin. Examples of suitable acetic acid NSAID derivatives may include Aceclofenac; Acemetacin; Actarit; Alcofenac; Amfenac; Clometacin; Diclofenac; Etodolac; Felbinac; Fenclofenac; Indometacin; Ketorolac; Metiazinic acid; Mofezolac; Naproxen; Oxametacin; Sulindac; or Zomepirac. Examples of suitable enolic acid NSAID derivatives may include Piroxicam; Meloxicam; Tenoxicam; Droxicam; Lornoxicam; or Isoxicam. Examples of Fenamic acid NSAID derivatives may include Mefenamic acid; Meclofenamic acid; Flufenamic acid; or Tolfenamic acid.

In embodiments where the NSAID is a cyclooxygenase (COX) inhibitor, it may be either a cyclooxygenase 1 (COX 1) inhibitor, or a cyclooxygenase 2 (COX 2) inhibitor. Examples of suitable COX inhibitors may include Celecoxib; Etoricoxib; Lumiracoxib;

Meloxicam; Rofecoxib; or Valdecoxib.

The non-steroidal anti-inflammatory drug may be selected from a group consisting of: Alminoprofen; Benoxaprofen; Dexketoprofen; Flurbiprofen; Ibuprofen; Indoprofen; Ketoprofen; Loxoprofen; Pranoprofen; Protizinic acid; Suprofen; Aceclofenac; Acemetacin; Actarit; Alcofenac; Amfenac; Clometacin; Diclofenac; Etodolac; Felbinac; Fenclofenac; Indometacin; Ketorolac; Metiazinic acid; Mofezolac; Naproxen;

Oxametacin; Sulindac; Zomepirac; Celecoxib; Etoricoxib; Lumiracoxib; Meloxicam; Rofecoxib; Valdecoxib; Aloxipirin; Aminophenazone; Antraphenine; Aspirin; Azapropazone; Benorilate; Benzydamine; Butibufen; Chlorthenoxacin; Choline Salicylate; Diflunisal; Emorfazone; Epirizole; Feclobuzone; Fenbufen; Glafenine; Hydroxylethyl salicylate; Lactyl phenetidin; Mefenamic acid; Metamizole;

Mofebutazone; Nabumetone; Nifenazone; Niflumic acid; Phenacetin; Pipebuzone; Propyphenazone; Proquazone; Salicylamide; Salsalate; Tiaramide; Tinoridine; and Tolfenamic acid.

A preferred non-steroidal anti-inflammatory drug may be Alminoprofen, Benoxaprofen, Dexketoprofen, Flurbiprofen, Ibuprofen, Indoprofen, Ketoprofen, Loxoprofen, Pranoprofen protizininic acid, or Suprofen. Preferably, the NSAID is Ibuprofen.

The non-steroidal anti-inflammatory drug may be used in the form of a pharmaceutically acceptable salt, solvate, or solvate of a salt, e.g. the hydrochloride.

NSAIDs described herein may be provided as racemates, or as individual enantiomers, including the R- or S-enantiomer. Thus, the NSAID may comprise R-ibuprofen or S-ibuprofen, or a combination thereof.

The pharmaceutical composition may be used to treat a fulminant respiratory disorder. The composition may be used to treat oedema, i.e. fluid accumulation in the lungs. Oedema may be caused by the failure of the heart to remove fluid from the lung circulation (referred to as cardiogenic pulmonary oedema), or from a direct injury to the lung parenchyma (referred to as non-cardiogenic pulmonary oedema).

As described in the Examples, and as shown in FIGS. 3 and 4, the inventors have demonstrated, in an in vivo mouse model, that ibuprofen, when formulated in oil, may be used to prevent, treat or ameliorate the symptoms of respiratory diseases caused by viral infections. The inventors therefore believe that they are the first to demonstrate that ibuprofen can be used in the treatment of acute and chronic viral infections.

A common pathogen-induced respiratory disorder, or acute respiratory distress, is hospital- and community-acquired pneumonia. Pneumonia is characterised by cough, chest pains, fever, and difficulty in breathing due to pulmonary oedema. These symptoms occur in all pneumonia patients regardless of the pathogen that causes the pneumonia, which can be bacterial (e.g. Streptococcus pneumonia), viral (e.g. influenza virus) and fungal (e.g. Histoplasma capsulatum). Regardless of the pathogen causing pneumonia, the symptoms are the same and the inflammatory processes regardless of the stimulus cause exaggerated inflammatory responses, resulting in potentially fatal pulmonary oedema. In the animal models of respiratory disorders associated with the influenza infection (i.e. a viral pathogen) described in the Examples, the end points are designed to measure pulmonary oedema related end points (i.e. post infection survival). The effect on post infection survival for the compositions of the invention, in the influenza assay, supports the likelihood for effects in pulmonary oedema caused by any type of pathogen, be it viral, bacterial or fungal.

Accordingly, the inventors believe that the compositions described herein may be used to combat respiratory disorders (i.e. oedema) that are caused by any microbial or pathogenic infection, such as bacterial, fungal or viral (e.g. acute viral infections), and which, in some cases (e.g. influenza infections), can cause death. The compositions may be used as a prophylactic (to prevent the development of a respiratory disorders associated with microbial infection), or they may be used to treat existing respiratory disorders associated with microbial infections.

Examples of micro-organisms, which may cause a respiratory disorder, which may be treated with compositions according to the invention, may include bacteria, viruses, fungi, or protozoa, and other pathogens and parasites, which can cause respiratory disorders. These pathogens can cause upper or lower respiratory tract diseases, or obstructive or restrictive lung diseases, each of which may be treated. The most common upper respiratory tract infection is the common cold, which may be treated. In addition, infections of specific organs of the upper respiratory tract, such as sinusitis, tonsillitis, otitis media, pharyngitis and laryngitis are also considered as upper respiratory tract infections, which may be treated with the compositions described herein.

The most common lower respiratory tract infection is pneumonia, which may be treated with the compositions described herein. Pneumonia is usually caused by bacteria, particularly Streptococcus pneumoniae. However, tuberculosis is also an important cause of pneumonia. Other pathogens, such as viruses and fungi, can also cause pneumonia, for example Severe Acute Respiratory Distress, Acute Respiratory Distress Syndrome and pneumocystis pneumonia. Therefore, the compositions of the invention may be used to treat Respiratory Distress Syndrome (RDS), Acute Respiratory Distress

Syndrome (ARDS), or Acute Lung Injury (ALI). In addition, the compounds may be used to treat diseases with concomitant pathogen infection such as chronic obstructive pulmonary disorder, cystic fibrosis and bronchiolitis.

The pharmaceutical composition of the invention may be useful for preventing, treating and/or ameliorating a respiratory disorder caused by a bacterial infection. The bacterium causing the infection may be a Gram-positive bacterium or a Gram-negative bacterium. Examples of bacteria, which may cause a respiratory disorder, against which the compositions are effective, may be selected from a list consisting of: Streptococcus spp., Staphylococcus spp., Haemophilus spp., Klebsiella spp., Escherichia spp., Pseudomonas spp., Moraxella spp., Coxiella spp., Chlamydophila spp., Mycoplasma spp., Legionella spp. and Chlamydia spp. Species of bacteria, which may cause a respiratory disorder, against which the compositions in accordance with the invention are effective, may be selected from a list consisting of: Streptoccoccus pneumoniae, Staphylococcus aureus, Haemophilus influenzae, Klebsiella pneumoniae, Escherichia coli, Pseudomonas aeroginosa, Moraxella catarrhalis, Coxiella burnettie, Chlamydophila pneumoniae, Mycoplasma pneumoniae, Legionella pneumophila and Chlamydia trachomatis.

The compositions may also be useful for preventing, treating and/or ameliorating a respiratory disorder caused by a fungal infection. Examples of fungi, which may cause a respiratory disorder, against which the compositions are effective, may be selected from a group consisting of: Histoplasma spp., Blastomyces spp., Coccidioides spp., Cryptococcus spp., Pneumocystis spp. and Aspergillus spp. Species of fungi, which may cause a respiratory disorder, against which the compositions are effective, may be selected from a group consisting of: Histoplasma capsulatum, Blastomyces, Coccidioides immitis, Cryptococcus neoformans, Pneumocystis jiroveci, Aspergillus flavus, Aspergillus fumigatus, Aspergillus nidulans, Aspergillus niger, Aspergillus parasiticus and Aspergillus terreus.

The compositions of the invention may be particularly useful for preventing, treating and/or ameliorating a respiratory disorder caused by a viral infection. The inventors believe that the compositions of the invention may be used in the treatment of any number of acute or chronic viral infections, and respiratory disorders which may result therefrom. The compositions may be used as a prophylactic (to prevent the development of a viral infection) or may be used to treat existing viral infections. In one embodiment, the composition may be used to treat a viral infection, which may be chronic, but which is preferably an acute viral infection.

The virus may be an enveloped virus. The virus may be an RNA virus or a retrovirus. For example, the viral infection, which may be treated, may be a paramyxovirus or an orthomyxovirus infection. The virus causing the infection may be a poxvirus, iridovirus, thogavirus, or torovirus. The virus causing the infection may be a Filovirus, arenavirus, bunyavirus, or a rhabdovirus. It is envisaged that the virus may be a hepadnavirus, coronavirus, or a flavivirus. In particular, the following viral infections linked to respiratory complications may be treated: Respiratory syncytial virus, Human bocavirus, Human parvovirus B19, Herpes simplex virus 1, Varicella virus, Adenovirus, Parainfluenza virus, Enterovirus 71, Hantavirus, SARS virus, SARS-associated coronavirus, Sin Nombre virus, Respiratory reovirus, Haemophilus influenza or Adenovirus.

The invention extends to the treatment of infections with derivatives of any of the viruses disclosed herein. The term “derivative of a virus” can refer to a strain of virus that has mutated from an existing viral strain.

The virus may be selected from the group of viral genera consisting of Influenzavirus A; Influenzavirus B; Influenzavirus C; Isavirus and Thogotovirus, or any derivative of the foregoing viruses. Influenza viruses A-C include viruses that cause influenza in vertebrates, including birds (i.e. avian influenza), humans, and other mammals. Influenzavirus A causes all flu pandemics and infects humans, other mammals and birds. Influenzavirus B infects humans and seals, and Influenzavirus C infects humans and pigs. Isaviruses infect salmon, and thogotoviruses infect vertebrates (including human) and invertebrates.

Thus, compositions of the invention may be used to treat an infection of any of Influenzavirus A, Influenzavirus B, or Influenzavirus C, or a derivative thereof. It is preferred that the compositions may be used for treating an infection of Influenza A, or a derivative thereof. Influenza A viruses are classified, based on the viral surface proteins hemagglutinin (HA or H) and neuraminidase (NA or N). Sixteen H subtypes (or serotypes) and nine N subtypes of influenza A virus have been identified. Thus, the compositions of the invention may be used to treat an infection of any serotype of Influenzavirus A selected from the group of serotypes consisting of: H1N1; H1N2; H2N2; H3N1; H3N2; H3N8; H5N1; H5N2; H5N3; H5N8; H5N9; H7N1; H7N2; H7N3; H7N4; H7N7; H9N2; and H1ON7, or a derivative thereof. The inventors believe that compositions of the invention may be particularly useful for treating viral infections of H1N1 virus, or a derivative thereof. It will be appreciated that swine flu is a strain of the H1N1 virus.

The inventors have found that, following infection with a virus, IFN-γ and TNF-α can cause fluid to leak into the lungs of an infected subject, which results in respiratory disorders that can cause eventual death. Although they do not wish to be bound by hypothesis, the inventors believe that the compositions of the invention may be used to treat viral infections because they can act as an inhibitor of cytokine production, and in particular IFN-γ and/or TNF-α, and that, therefore, they can be used to treat the respiratory disorder caused by a viral infection.

The compounds of the invention may therefore be used to ameliorate inflammatory symptoms of virally-induced cytokine production. The anti-inflammatory composition may have an effect on any cytokine. However, preferably it modulates IFN-γ and/or TNF-α. The compositions may be used to treat inflammation in an acute viral infection of a naïve subject. The term “naïve subject” can refer to an individual who has not previously been infected with the virus. It will be appreciated that once an individual has been infected with a virus such as herpes, that individual will always retain the infection.

It is especially intended that the compositions may be used to treat the final stages of a viral infection, such as the end stages of influenza. The compositions may also be used to treat a viral flare-up. A viral flare-up can refer to either the recurrence of disease symptoms, or an onset of more severe symptoms.

It will be appreciated that the compositions described herein may be used to treat microbial (e.g. viral) infections in a monotherapy (i.e. use of the pharmaceutical compositions of the first aspect alone). Alternatively, the compositions of the invention may be used as an adjunct to, or in combination with, known antimicrobial therapies. For example, conventional antibiotics for combating bacterial infections include amikacin, amoxicillin, aztreonam, cefazolin, cefepime, ceftazidime, ciprofloxacin, gentamicin, imipenem, linezolid, nafcillin, piperacillin, quinopristin-dalfoprisin, ticarcillin, tobramycin, and vancomycin. In addition, compounds used in antiviral therapy include acyclovir, gangcylovir, ribavirin, interferon, nucleotide or non-nucleoside inhibitors of reverse transcriptase, protease inhibitors and fusion inhibitors. Furthermore, conventional antifungal agents include, for example farnesol, clotrimazole, ketoconazole, econazole, fluconazole, calcium or zinc undecylenate, undecylenic acid, butenafine hydrochloride, ciclopirox olaimine, miconazole nitrate, nystatin, sulconazole, and terbinafine hydrochloride. Hence, compositions according to the invention may be used in combination with such antibacterial, antiviral and antifungal agents.

The compositions of the invention may have a number of different forms provided that it is orally administerable. The composition may be administered orally either in liquid or solid composition form. Compositions suitable for oral administration include solid forms, such as pills, capsules, granules, tablets, and powders, and liquid forms, such as solutions, syrups, elixirs, aerosols for administration via the mouth, sprays, micellar solutions, liposome suspensions, or any other suitable form for oral administration to a subject (person or animal) in need of treatment. It will be appreciated that the vehicle for medicaments according to the invention should be one which is well-tolerated by the subject to whom it is given, and enables delivery of the NSAID directly to the site infected by the pathogen (i.e. the virus, bacterium or fungus), such as the lungs, in order to treat a respiratory disease.

It will be appreciated that the amount of NSAID in the composition that is required is determined by its biological activity and bioavailability, which in turn depends on the physicochemical properties of the NSAID, and whether it is being used as a monotherapy, or in a combined therapy. The frequency of administration will also be influenced by the above-mentioned factors and particularly the half-life of compounds within the subject being treated.

Optimal dosages to be administered may be determined by those skilled in the art, and will vary with the particular NSAID in use, the strength of the preparation, and the advancement of the disease condition. Additional factors depending on the particular subject being treated will result in a need to adjust dosages, including subject age, weight, gender, diet, and time of administration.

It will be appreciated that a skilled person will be able to calculate required doses, and optimal concentrations of the NSAID at a target tissue, based upon the pharmacokinetics of the chosen compound. Known procedures, such as those conventionally employed by the pharmaceutical industry (eg in vivo experimentation, clinical trials, etc.), may be used to establish specific formulations of the compounds of the invention and precise therapeutic regimes (such as daily doses of the compounds and the frequency of administration).

Generally, the maximum over-the-counter (OTC) daily dose of ibuprofen that is available to patients for treating most conditions is 1200 mg ibuprofen/day. However, patients suffering from certain diseases, such as cystic fibrosis for example, may be prescribed, by a physician, a maximum of 800 mg of ibuprofen administered four times a day (i.e. a maximum daily dose of 3200 mg/day), as such high doses can have a positive effect in reducing the symptoms of these diseases (e.g. CF). However, a significant problem with such high doses of ibuprofen and other NSAIDs, which is why they are prescription only, is that treated patients suffer from the side-effect of gastric ulceration or gut erosion, as well as nausea, diarrhoea, headaches and hypertension.

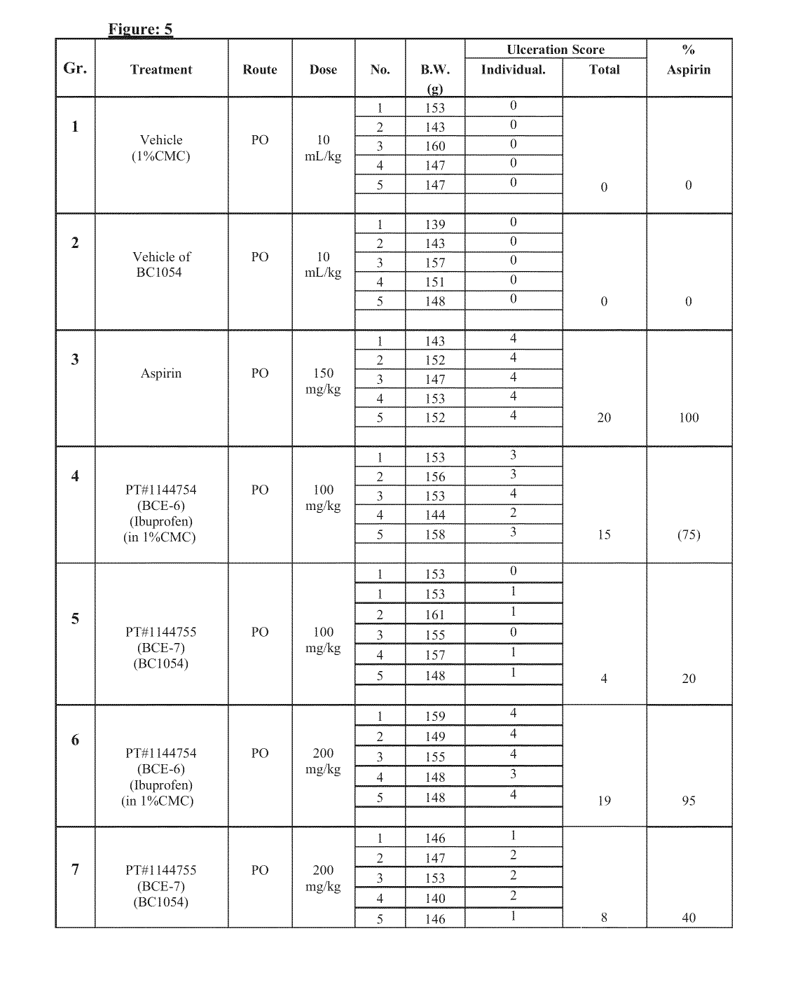

As described in Example 2, and as illustrated in FIG. 5, the inventors were very surprised to observe, in their in vivo rat models, that rats treated with massive doses of ibuprofen formulated in the lipid/ethanol vehicle used in the composition of the first aspect (i.e. lipid/alcohol) were surprisingly resistant to gastric ulceration. Indeed, ibuprofen doses of 100 mg/kg and 200 mg/kg administered to the rats described in Example 2 equate to a human equivalent dose (HED) of 7000 mg and 14000 mg, both of which showed limited gut erosion in the rat (compared to the current maximum daily human dose of 3200 mg ibuprofen, as discussed above). Advantageously, therefore, the compositions of the invention may be administered to patients requiring treatment with a high dose of NSAID (e.g. i.e. over 3200 mg/day) but avoid the deleterious side-effects of gut erosion that would be caused by the NSAID. This means that the compositions can be given for extended periods of time and/or at high doses to patients who would otherwise be susceptible to this side-effect.

Accordingly, generally, a daily dose of between 0.001 μg/kg of body weight and 200 mg/kg of body weight NSAID may be used for the prevention and/or treatment of a respiratory disorder (e.g. one which may be caused by a microbial (e.g. viral) infection) depending upon which compound is used. Suitably, a daily dose of between 0.001 μg/kg of body weight and 150 mg/kg of body weight, or between 0.001 μg/kg of body weight and 100 mg/kg of body weight, or between 0.01 μg/kg of body weight and 100 mg/kg of body weight, or between 0.1 μg/kg of body weight and 100 μg/kg of body weight, or between 0.01 μg/kg of body weight and 80 mg/kg of body weight of NSAID may be used.

Suitably, a daily dose of between 0.1 μg/kg of body weight and 65 mg/kg of body weight, or between approximately 0.1 μg/kg of body weight and 50 mg/kg of body weight, or between 0.001 μg/kg of body weight and 20 mg/kg of body weight, or between 0.01 μg/kg of body weight and 10 mg/kg of body weight, or between 0.01 μg/kg of body weight and 1 mg/kg of body weight, or between 0.1 μg/kg of body weight and 10 μg/kg of body weight of the NSAID may be used.

Daily doses of the NSAID may be given as a single administration (e.g. a single daily tablet or capsule). A suitable daily dose may be between 0.07 μg and 14000 mg (i.e. assuming a body weight of 70 kg), or between 0.70 μg and 10000 mg, or between 0.70 μg and 7000 mg, or between 10 mg and 3200 mg. A suitable daily dose may be between 0.07 μg and 700 mg, or between 0.70 μg and 500 mg, or between 10 mg and 450 mg. The composition may be administered before or after infection with the pathogen causing the respiratory disorder, such as the virus. The composition may be administered within 2, 4, 6, 8, 10 or 12 hours after infection. The composition may be administered within 14, 16, 18, 20, 22, or 24 hours after infection. The composition may be administered within 1, 2, 3, 4, 5, or 6 days after infection, or at any time period therebetween.

In embodiments where the infection being treated is an infection of influenza, independently of whether or not the influenza is a pandemic influenza, the subject is someone treated with compositions of the invention in whom symptoms of respiratory difficulty arise and/or in whom cytokine levels (any of the above mentioned cytokines, but typically IFN-α or TNF-γ) increase at the onset of symptoms of respiratory difficulty. More preferably, the subject is a subject in whom symptoms of respiratory difficulty arise, and/or in whom cytokine levels increase, at the following times after onset of influenza symptoms: from 12, 24, 18 or 36 hours or more (more preferably from 48 hours or more, from 60 hours or more, or from 72 hours or more; most preferably from 36-96 hours, from 48-96 hours, from 60-96 hours or from 72-96 hours). Alternatively, and independently of whether or not the influenza is a pandemic influenza, the subject is someone in whom symptoms of respiratory difficulty arise and/or in whom cytokine levels increase, at the onset (or early stage) of recruitment of the adaptive immune system into the infected lung.

It is envisaged that the compositions of the invention may be orally administered more than once to a subject in need of treatment. The composition may require administration twice or more times during a day. As an example, the composition may be administered as two (or more depending upon the severity of the viral infection being treated) daily doses of between 0.07 μg and 14000 mg, or between 0.07 μg and 7000 mg, or between 0.07 μg and 700 mg (i.e. assuming a body weight of 70 kg). A patient receiving treatment may take a first dose upon waking and then a second dose in the evening (if on a two dose regime) or at 3- or 4-hourly intervals thereafter, and so on. It is envisaged that the composition may be administered every day (more than once if necessary) following pathogenic infection. Thus, the compositions of the invention are preferably suitable for administration to a subject as described above, preferably suitable for administration at the aforementioned points after the onset of influenza symptoms.

A “therapeutically effective amount” of an NSAID is any amount which, when administered to a subject, provides prevention and/or treatment of a microbial infection, such as an acute viral infection.

For example, a therapeutically effective amount of the NSAID may be from about 0.07 μg to about 14000 mg, or from about 0.07 μg to about 10000 mg, or from about 0.07 μg to about 7000 mg, and preferably from about 0.7 μg to about 4800 mg. The amount of the NSAID may be from about 7 μg to about 3200 mg, or from about 7 μg to about 1200 mg. The amount of NSAID may alternatively be from about 0.07 μg to about 1500 mg, or from about 0.07 μg to about 700 mg, and preferably from about 0.7 μg to about 70 mg. The amount of the NSAID may be from about 7 μg to about 7 mg, or from about 7 μg to about 700 μg.

As discussed above, it is currently not possible to prescribe ibuprofen at a dose of more than 3200 mg/day due to the deleterious gut erosion side effects discussed above. However, the inventors have now surprisingly shown in FIG. 5, that rats treated with massive doses of ibuprofen (i.e. 100 mg/kg and 200 mg/kg ibuprofen in a rat equate to human equivalent doses (HED) of 7000 mg and 14000 mg, respectively) formulated in the lipid/ethanol vehicle of the invention were highly resistant to gut ulceration. Thus, unlike currently available formulations of NSAIDs, the compositions of the invention are non-gut erosive, and so allow a previously high and normally gut erosive dose of an NSAID, such as ibuprofen (i.e. 3200 mg/day), to be administered to patients with no concern to pain physicians. Accordingly, the compositions of the invention comprising an NSAID and a pharmaceutically acceptable vehicle comprising a lipid and an alcohol have profound analgesic characteristics, i.e. as a supra-analgesic for use in treating any inflammatory pain, such as rheumatoid arthritis or osteoarthritis, and not only patients suffering from respiratory disorders, such as CF.

Hence, in a fourth aspect, there is provided a pharmaceutically acceptable vehicle comprising a lipid and an alcohol in an orally administrable pharmaceutical composition comprising a non-steroidal anti-inflammatory drug (NSAID) or a derivative thereof, for use in the treatment of inflammatory pain, by oral administration of a dose of the NSAID or the derivative thereof of greater than 3200 mg/day.

In a fifth aspect of the invention, there is provided an orally administrable analgesic composition comprising a therapeutically effective amount of a non-steroidal anti- inflammatory drug (NSAID) or a derivative thereof, and a pharmaceutically acceptable vehicle comprising a lipid and an alcohol, for use in the treatment of inflammatory pain, by oral administration of a dose of the NSAID or the derivative thereof of greater than 3200 mg/day.

In a sixth aspect, there is provided a method of treating inflammatory pain, the method comprising orally administering, to a subject in need of such treatment, either (i) a pharmaceutically acceptable vehicle comprising a lipid and an alcohol in an orally administrable pharmaceutical composition comprising a non-steroidal anti-inflammatory drug (NSAID) or a derivative thereof, or (ii) an orally administrable analgesic composition comprising an NSAID or a derivative thereof, and a pharmaceutically acceptable vehicle comprising a lipid and an alcohol, wherein the method comprises administering, to the subject, a dose of the NSAID or the derivative thereof of greater than 3200 mg/day.

Advantageously, the compositions of the invention enable physicians to prescribe NSAIDs, such as ibuprofen, at doses higher than 3200 mg/day. In particular, the compositions may be administered to a patient who is susceptible to deleterious side-effects that are associated with taking high concentrations of an NSAID, i.e. more than 3200 mg/day, such as gut erosion. For example, the compositions may be administered at a daily dose of NSAID or derivative thereof, which is higher than 3300 mg/day, 3400 mg/day, 3500 mg/day, 4000 mg/day, 4500 mg/day, 5000 mg/day, 6000 mg/day, 7000 mg/day, 8000 mg/day, 9000 mg/day, 10 g/day, 11 g/day, 12 g/day, 13 g/day, or 14 g/day or more. Advantageously, as shown in FIG. 5, such higher doses of the NSAID avoid gastric ulceration.

Daily doses of the NSAID or derivative thereof may be given as a single administration (e.g. a single daily tablet or capsule). A suitable daily dose may be between greater than 3200 mg and 14000 mg (i.e. assuming a body weight of 70 kg), or between greater than 3200 mg and 10000 mg, or between greater than 3200 mg and 7000 mg, or between greater than 3200 mg and 5000 mg. A suitable daily dose may be between greater than 4000 mg and 14000 mg, or between greater than 4000 mg and 10000 mg, or between greater than 4000 mg and 7000 mg.

It is envisaged that the compositions of the invention may be orally administered more than once to a subject in need of treatment. The compositions may require administration twice or more times during a day. As an example, the composition may be administered as two or more daily doses of between greater than 3200 mg and 7000 mg, or between greater than 3200 mg and 5000 mg, or between greater than 3200 mg and 4000 mg (i.e. assuming a body weight of 70 kg).

In addition, since gut erosion is avoided at these higher NSAID doses, it will become possible for the current controls and supervision by doctors at these higher doses to be removed, and so these compositions will become over the counter (OTC) medicines, and not require prescription. Accordingly, this will provide higher dose, and greater efficacy products, to a large patient population. Conversely, the compositions of the invention may also be administered at lower doses (i.e. between 1600 mg/day and 3200 mg/day), and yet still achieve the same analgesic effect that would be achieved with higher doses of known NSAID compositions, while advantageously reducing the risk that the patient will suffer from gastric erosion. Due to the safety of using such higher doses of NSAIDs, which are currently available under prescription only, there would now be no need for these compositions to be made available only under prescription, and so they may be obtained over-the-counter.

Thus, in a seventh aspect, there is provided a pharmaceutically acceptable vehicle comprising a lipid and an alcohol in an over-the-counter (OTC), orally administrable pharmaceutical composition comprising a non-steroidal anti-inflammatory drug (NSAID) or a derivative thereof, for use in the treatment of inflammatory pain, by oral administration of a dose of the NSAID or the derivative thereof of greater than 1600 mg/day.

In an eighth aspect of the invention, there is provided an over-the-counter (OTC), orally administrable analgesic composition comprising a therapeutically effective amount of a non-steroidal anti-inflammatory drug (NSAID) or a derivative thereof, and a pharmaceutically acceptable vehicle comprising a lipid and an alcohol, for use in the treatment of inflammatory pain, by oral administration of a dose of the NSAID or the derivative thereof of greater than 1600 mg/day.

The dose of NSAID or derivative thereof may be between 1600 mg/day and 3200 mg/day. It will be appreciated that the compositions of the seventh and eighth aspects may be administered at any of the doses described herein, provided it is greater than 1600 mg/day.

Preferably, the NSAID is a profen, for example ibuprofen.

The compositions of the invention may be used to treat or relieve inflammatory pain in a wide variety of disease conditions, for example arthritis (e.g. rheumatoid arthritis or osteoarthritis), inflammatory bowel disease, endometriosis, pelvic inflammatory disease, ankylosing spondylitis, psoriatic arthritis, psoriasis or cystic fibrosis.

A “subject” can be a vertebrate, mammal, or domestic animal, and is preferably a human being. Hence, compositions according to the invention may be used to treat any mammal, for example human, livestock, pets, or may be used in other veterinary applications.

A “pharmaceutically acceptable vehicle” as referred to herein can be any combination of compounds known to those skilled in the art to be useful in formulating pharmaceutical compositions, but which comprises a lipid (e.g. at least 30% (w/w)) and an alcohol.

In one embodiment, the pharmaceutically acceptable vehicle described herein may be a solid, and the pharmaceutical composition may be in the form of a powder or tablet. In addition to the lipid component and alcohol, a solid pharmaceutically acceptable vehicle may comprise one or more substances which may also act as flavouring agents, lubricants, solubilisers, suspending agents, dyes, fillers, glidants, compression aids, inert binders, sweeteners, preservatives, dyes, coatings, or tablet-disintegrating agents. The vehicle may also be an encapsulating material. In powders, the vehicle may be a finely divided solid that is in admixture with the finely divided active agent (i.e. the NSAID). In tablets, the active agent may be mixed with a vehicle having the necessary compression properties in suitable proportions and compacted in the shape and size desired. Suitable solid vehicles may comprise, for example calcium phosphate, magnesium stearate, talc, sugars, lactose, dextrin, starch, gelatin, cellulose, polyvinylpyrrolidine, low melting waxes and ion exchange resins.

In a preferred embodiment, the pharmaceutical vehicle may be a liquid, and the pharmaceutical composition may be in the form of a solution. Liquid vehicles are used in preparing solutions, suspensions, emulsions, syrups, elixirs and pressurized compositions. The active compound may be dissolved or suspended in a pharmaceutically acceptable liquid vehicle such as water (though it is preferred that the vehicle does not comprise water), an organic solvent, a mixture of both, or pharmaceutically acceptable oils or fats. In addition to the lipid component, the liquid vehicle may also comprise other suitable pharmaceutical additives such as solubilisers, emulsifiers, buffers, preservatives, sweeteners, flavouring agents, suspending agents, thickening agents, colours, viscosity regulators, stabilizers or osmo-regulators. Suitable examples of liquid vehicles for oral administration may include water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). The vehicle can also be an oily ester, such as ethyl oleate or isopropyl myristate.

The composition is preferably administered orally in the form of a sterile solution or suspension containing other solutes or suspending agents (for example, enough saline or glucose to make the solution isotonic), bile salts, acacia, gelatin, sorbitan monoleate, polysorbate 80 (oleate esters of sorbitol and its anhydrides copolymerized with ethylene oxide), and the like.

However, the composition may or may not comprise a surfactant. Examples of surfactants which may or not be included in the composition include a phospholipid, such as phosphatidylcholine (lecithin) and phosphatidyl ethanolamine; soaps and detergents, including fatty alkali metal, ammonium, and triethanolamine salts, and detergents, including (a) cationic detergents such as, dimethyl dialkyl ammonium halides, and alkyl pyridinium halides; (b) anionic detergents such as alkyl, aryl, and olefin sulfonates, alkyl, olefin, ether, and monoglyceride sulfates, and sulfosuccinates; (c) non-ionic detergents such as fatty amine oxides, fatty acid alkanolamides, and polyoxyethylenepolypropylene copolymers; and (d) amphoteric detergents such as alkyl-b-aminopropionates, and 2-alkyl-imidazoline quaternary ammonium salts. Another example of a detergent may include sodium dodecyl sulphate dimethyl sulphoxide. Preferably, the vehicle of the invention does not comprise any of these surfactants.

The inventors believe that the pharmaceutically acceptable vehicle may preferably comprise at least 30% (w/w) lipid, possibly in the absence of ethanol.

Thus, in a further aspect, there is provided a pharmaceutical composition for oral administration, the composition comprising a therapeutically effective amount of a non-steroidal anti-inflammatory drug (NSAID) or a derivative thereof, and a pharmaceutically acceptable vehicle comprising at least 30% (w/w) lipid, wherein the composition is for use in treating a respiratory disorder.

In another aspect, there is provided a method of preventing, treating and/or ameliorating a respiratory disorder, the method comprising orally administering, to a subject in need of such treatment, a pharmaceutical composition comprising a therapeutically effective amount of a non-steroidal anti-inflammatory drug (NSAID) or a derivative thereof, and a pharmaceutically acceptable vehicle comprising at least 30% (w/w) lipid.

In another aspect, there is provided a use of a pharmaceutically acceptable vehicle comprising at least 30% (w/w) lipid in an orally administrable pharmaceutical composition, for increasing the bioavailability of a non-steroidal anti-inflammatory drug (NSAID) or a derivative thereof in a subject's lung.

All of the features described herein (including any accompanying claims, abstract and drawings), and/or all of the steps of any method or process so disclosed, may be combined with any of the above aspects in any combination, except combinations where at least some of such features and/or steps are mutually exclusive.

Embodiments of the invention will now be further described, by way of example only, with reference to the following Examples, and to the accompanying diagrammatic drawings, in which:

FIG. 1 is a graph showing the results of an in vivo mouse challenge (measuring % weight loss), in which mice were first infected with a H1N1 virus, and then, on day 3 post-challenge animals were intraperitoneally injected with ibuprofen at a dose of 335.6 μg/animal in 10 μDMSO (equivalent to 20 mg/kg/day; i.e. 1200 mg per person day as maximum standard dose). The control mice received the intraperitoneal drug vehicle only, i.e. 10 μl DMSO. The percentage weight loss was measured over the course of 6 days;

FIG. 2 is a graph showing the survival rate of mice in the in vivo mouse challenge described in relation to FIG. 1. The mice influenza-challenged mice were intraperitoneally injected with ibuprofen as a single dose on day 3, and the percentage rate of survival was measured. No ibuprofen was added to the mice of the control, just the IP vehicle (10 μl DMSO);

FIG. 3 is a graph showing the results of an in vivo mouse challenge (% weight loss), in which mice were infected with a H1N1 virus, and then on day 3 post-challenge animals received an oral dosage of ibuprofen at a dose of 335.6 μg/animal in a lipid vehicle, i.e. an oral gavage of ibuprofen in 100 μl of 10% Ethanol, 90% rapeseed oil (known herein as BC1054). The control mice received an oral dosage of just the oral drug vehicle, i.e. 100 μl of 10% Ethanol, 90% rapeseed oil. The percentage weight loss was measured over the course of 6 days;

FIG. 4 is a graph showing the survival rate of mice in the in vivo mouse challenge described in relation to FIG. 3. The mice were orally administered with ibuprofen as a single dose on day 3, and the percentage rate of survival was measured. No ibuprofen was added to the mice of the control;

FIG. 5 is a table showing gastric irritation in rats. Vehicle and test compounds (Groups 1-7) were each administered orally (PO) to fasted rats. Each group included five animals. Group 1 were treated with 10 mL/kg of 1% carboxymethylcellulose (CMC) vehicle with no ibuprofen; Group 2 were treated with 10 mL/kg of only the vehicle of BC1054, i.e. 10% Ethanol, 90% rapeseed oil, and no ibuprofen; Group 3 were treated with 150 mg/kg aspirin; Group 4 were treated with 100 mg/kg of ibuprofen dissolved in 1% CMC; Group 5 were treated with 100 mg/kg of ibuprofen dissolved in 10% Ethanol, 90% rapeseed oil (i.e. BC1054); Group 6 were treated with 200 mg/kg of ibuprofen in 1% CMC; and Group 7 were treated with 200 mg/kg of ibuprofen dissolved in 10% Ethanol, 90% rapeseed oil (i.e. BC1054). The animals were sacrificed four hours after dosing and gastric mucosal lesions were scored. A score of 50 percent or more (≧50%) relative to the aspirin-treated group (150 mg/kg PO, set as 100%) is considered positive in gastric irritation and is shown in parenthesis; and

FIG. 6 is a bar chart comparing the relative concentration of ibuprofen found in the lung of test mice that had been treated with BC1054 (left hand bar) and control mice that had been treated with normal ibuprofen (i.e. not in a lipid vehicle).

### EXAMPLES

The inventors carried out a range of in vivo mouse experiments in order to determine the effects of ibuprofen on influenza-challenged mice when administered orally in an oily/lipid vehicle (known herein as BC1054), or when administered intraperitoneally. The inventors have convincingly demonstrated in the results described below that ibuprofen, when administered orally in an oil-based formulation, results in a considerable reduction in the viral symptoms (i.e. reduction in weight loss, and increase in survival rate), but not when administered intraperitoneally. The inventors also investigated whether or not the composition of the invention (BC1054) eroded the gut of rats in vivo, and determined that it exhibited reduced ulceration effects. Finally, the inventors also determined the in vivo concentration of ibuprofen in the lungs of mice treated with BC1054, i.e. its bioavailability.

### Materials and Methods

### In Vivo Mouse Studies

### Protocol:

Five groups (n=10) of C57BLK/6 female mice (6-7 weeks old), were divided into five experimental groups containing ten animals each. On day 1, animals received an intranasal lethal dose (50 μl total, 25 μl nostril) of Influenza A/PR/8/34 under halothane induced anaesthesia.

On day 3, post-challenge with the virus, the animals received the following treatments:
Group A was intraperitoneally injected with ibuprofen at a dose of 335.6 μg/animal in 10 μl DMSO (equivalent to 20 mg/Kg/day; i.e. 1200 mg per person per day as maximum standard dose);
Group B received an oral gavage of ibuprofen at the same dose as group A but dissolved in 100 μl of 10% Ethanol; 90% rapeseed oil (an embodiment of the composition of the invention referred to herein as the formulation BC1054); and
Group C animals 1-5 received vehicle control (IP 10 μl DMSO) and animals 6-10 vehicle control (gavage of 10% Ethanol; 90% rapeseed oil).

The animals were weighed, and monitored for signs of infection daily up to day 6 when all animals were culled. FIGS. 1-4 represent the average weight loss per group and animal survival.

### Rat Gastric Irritation in Vivo Experiments

Seven groups of rats, each group consisting of five animals, were each administered orally (PO) with test formulations and control compounds. Group 1 animals were treated with 10 mL/kg of 1% carboxymethyl cellulose (CMC) vehicle with no ibuprofen, and Group 2 were treated with 10 mL/kg of only the vehicle of the BC1054 formulation, i.e. 10% Ethanol, 90% rapeseed oil. Hence, no ibuprofen was administered to this group. Group 3 animals were treated with 150 mg/kg aspirin, and Group 4 animals were treated with 100 mg/kg of ibuprofen dissolved in 1% CMC vehicle. Group 5 were treated with 100 mg/kg of ibuprofen dissolved in 10% Ethanol, 90% rapeseed oil (i.e. BC1054), and Group 6 were treated with 200 mg/kg of ibuprofen in 1% CMC. Finally, Group 7 animals were treated with 200 mg/kg of ibuprofen dissolved in 10% Ethanol, 90% rapeseed oil (i.e. BC1054).

The animals were sacrificed four hours after dosing with the test compound (or control vehicle) and gastric mucosal lesions were then scored according to the following criteria: 0=no lesions, 1=hyperemia, 2=one or two slight lesions, 3=more than two slight lesions or severe lesions, 4=very severe lesions (Herrerias et al., Dig. Dis. Sci., 2003). A score of 50 percent or more (≧50%) relative to the aspirin-treated group (150 mg/kg PO, set as 100%) was considered positive in gastric irritation and is shown in parenthesis in the table of FIG. 5.

### Determining in Vivo Ibuprofen Concentration in Mice

### Animals

Female and male C57BLK6 mice, aged 5 and 4 weeks, respectively, were supplied by Elevage Janvier. After arrival, the mice were allowed to acclimate for at least 7 days. Animals were housed in groups of three and had access to food and water ad libetum for the duration of the study and acclimation period. Mice were uniformly allocated to study to ensure that all cages were represented in the treatment groups.

### Study Protocol

Materials: Ibuprofen (suspension in water) and BC1054
Doses: 20 [mg/kg]
Treatment: single dose; p.o.
Application volume: 5 ml/kg body weight (bw)
Application timing: Application =T0
Animals per group: n=3

### Determination of the Analyte in Lungs

Four hours after dosing, mice were culled and lungs were removed, frozen and stored at −80° C. until required. Lung samples were ground in three volumes of acetonitrile (100 mg organ, 300 μL acetonitrile) and the precipitated protein was removed by centrifugation at 14000×g RCF for 10 minutes. The supernatant was transferred to a new tube and dried under vacuum for 120 minutes at 40° C. The dried residue was re-dissolved in 25 μL water per 50 mg tissue containing 0.01% ammonia V/V assisted by sonication and then subjected to centrifugation at 14000×g RCF for two minutes before being loaded into a glass vial for automated injection onto an HPLC system.

### HPLC System

HPLC separation was made with a gradient system with methanol (0.1% Ammonium formiate, pH 7.2) as the stronger eluant. The flow rate was 200 μL per minute using a 2 mm diameter, 50 mm reprosil C18 (Dr. Maisch, GmbH, Ammerbuch) column. Blank samples and QCs were run every 20 samples and the standard curve was repeated after sample runs. No carry over between samples of significance was observed.

### Examples

### In vivo Mouse and Rat Studies

### Example 1

### Viral Challenge Experiments

Using standard techniques as described above, mice were infected with a H1N1 virus which was allowed to become established in each of the subjects. Each test mouse was then treated with ibuprofen, either intraperitoneally (in DMSO) or orally (in the lipid/ethanol formulation, BC1054). The weight loss of both treated and untreated mice was then determined.

As shown in FIG. 1, the mice that received intraperitoneal doses of ibuprofen in DMSO showed about a 30% higher reduction in weight loss than in the control mice. Similarly, with reference to FIG. 2, the mice that received intraperitoneal doses of ibuprofen in DMSO had a lower percentage survival rate than the control mice, especially after day four. Thus, from these data, the inventors believe that intraperitoneal doses of ibuprofen did not show any beneficial effect on influenza challenged mice.

Referring now to FIG. 3, the mice that received oral doses of ibuprofen dissolved in lipid (i.e. the composition known herein as BC1054) surprisingly showed about a 20% lower reduction in weight loss than in the control mice, this effect becoming particularly apparent by day 6. Similarly, with reference to FIG. 2, the mice that received oral doses of ibuprofen in the BC1054 formulation had a 20% higher percentage survival rate than the control mice, especially after day 5. Accordingly, the inventors believe that orally administering ibuprofen in a lipophilic, oil-based vehicle, as described herein, has a marked benefit on the survival of the mice.

### Example 2

### Gastric Erosion Experiments in Rat

The table shown in FIG. 5 summarises the results of the gastric irritation experiments in rats. Aspirin, at a dose of 150 mg/kg, is known to be highly gut erosive in rat, as shown in the individual ulceration scores of “4” for each of the five animals, the total score being “20” (4×5), and so was set as the benchmark value of 100% against which the other formulations were compared. As expected, the two controls of vehicle only (Groups 1 and 2) showed no ulceration and so were scored “0”. However, Group 4 (i.e. 100 mg/kg) of ibuprofen in the vehicle of 1% CMC, showed significant ulceration, i.e. 75% compared to the aspirin ulceration score. Doubling the dose of ibuprofen to 200 mg/kg in 1% CMC vehicle increased the ulceration score to 95% that of aspirin.

The two test Groups 5 and 7, however, which were doses of 100 mg/kg and 200 mg/kg ibuprofen in the lipid formulation BC1054 (i.e. 10% ethanol and 90% rapeseed oil), respectively, showed ulceration scores of only 20% and 40% compared to the aspirin benchmark score. Both of these effects were considered to be indicative as non-gut erosive by the expert investigator. Accordingly, it is clear from these data that the composition of the invention, known herein as BC1054, shows surprisingly low levels of gut ulceration compared to the other formulations tested, especially aspirin. This was particularly surprising because 100 mg/kg ibuprofen in the rat equates to a human equivalent dose of 7 g/day, and 200 mg/kg ibuprofen in the rat equates to a human equivalent dose of 14 g/day. These are massive doses if one considers that a human is usually prescribed a maximum daily dose of between 1200 mg and 3200 mg/day ibuprofen. Accordingly, the inventors believe that the formulation of the invention has some known protective effect on the lining of the gut, such that incredibly high doses of ibuprofen may be administered to the rat (and hence human) without suffering the problem of gut ulceration and erosion.

### Example 3

### Determination of In Vivo Ibuprofen Concentration in Mice

With reference to FIG. 6, there is shown a relative comparison of the concentrations of ibuprofen found in the lungs of mice. As can be seen, the concentration of ibuprofen found in the lungs of the control mice (i.e. animals orally administered with ibuprofen in standard vehicle) was about 400 nmol. However, to their surprise, the concentration of ibuprofen in the lungs of the mice administered with the formulation of the invention (i.e. BC1054) was about 3250 nmol, i.e. approximately 8-fold higher. This was totally unexpected, and is a clear demonstration that the composition of the invention results in a significant increase in the bioavailability of the NSAID (ibuprofen in this case) in the lung.

### SUMMARY

In summary, the inventors were surprised to observe that ibuprofen, when administered orally in a lipophilic excipient (i.e. olive oil, rapeseed oil or linseed oil) significantly improved survival in influenza-challenged mice (see FIGS. 3 and 4), whereas the same dose of ibuprofen administered intraperitoneally showed no positive effect (see FIGS. 1 and 2). The encouraging results of the in vivo mouse studies described in the Examples clearly demonstrate that mice infected with a H1N1 virus can be effectively treated by administration of a single oral dose of ibuprofen present in an oily formulation. Hence, any NSAID, when formulated in a carrier having a high concentration of lipid, and orally administered, will result in a much higher bioavailability in the lung compared to intraperitoneal delivery or oral delivery using a non-lipid vehicle. That this is true is clearly demonstrated in FIG. 6, which shows that the concentration of ibuprofen in the lungs of mice orally administered with the lipid-based composition of the invention is 8-fold higher than in the lungs of mice that received an oral dose of ibuprofen present in a normal (i.e. non-lipid-based) vehicle.

Although not wishing to be bound by any theory, the inventors believe that this dramatic increase in bioavailability is achieved because, when a drug/lipid formulation is swallowed, the lipids are mixed with bile in the stomach, and form oil/NSAID micelles. These oil/NSAID micelles are then believed to be absorbed by epithelial cells of the proximal gut and converted in chylomicrons, which are then released into the lymphatic system, transported first to the central venous vasculature, and then rapidly to the heart, which pumps the NSAID-rich venous blood eventually to the lung. As a result, the NSAID is delivered in very high concentrations in oxygenated blood directly to the lung increasing its bioavailability at the treatment site. Clearly, achieving a high concentration of the NSAID, such as ibuprofen, in the lung (i.e. 8 times higher) will be particularly advantageous, when treating respiratory disorders, for example those caused by viral infections.

As discussed in Baumgarth and Kelso supra, Thi cytokines are key to the pathophysiology of over-reactive inflammatory response in the lung, to microbial pathogens. An important mechanism by which IFN-γ and TNF-α produce their inflammatory effect is by stimulating prostaglandin synthesis. Enhanced prostaglandin function results in vasoconstriction, oedema and neutrophil chemotaxis in the inflamed lung, which are very important in the pathogenesis of severe lung inflammatory indications such as pneumonia. Thus, therapies that reduce prostaglandin secretion, such as the administration of ibuprofen in the oily formulation of the invention, as described herein, at a sufficient therapeutic concentration, will prevent the downstream Thi initiated consequences of microbial pneumonia.

The inventors were very surprised to observe the low gut erosion data for the high concentrations of BC1054 that were tested, shown in FIG. 5, and believe that this may be explained by the fact that the formulation of the invention is capable of inhibiting prostaglandin secretion and activity. In addition, it is also postulated that the high lipid component in the formulation of the invention provides a physical protective barrier against the eroding effects of the NSAID. Thus, the inventors believe that the composition of the invention, not only increases bioavailability of the NSAID (e.g. ibuprofen) in the lung, possibly via the chylomicron route, but also prevents erosion of the gut, even at high doses (i.e. human equivalent doses of 7 g/day and 14g/day) by forming a physical barrier to the gastric mucosa.

Advantageously, BC1054 may therefore be administered to patients requiring treatment with high doses of ibuprofen (e.g. cystic fibrosis) and avoid the deleterious side-effects of gut erosion, meaning that the composition can be given for extended periods of time to patients who would otherwise be susceptible to this side-effect. There is therefore an “engorged therapeutic window”, i.e. a large distance between the dose of drug which is effective and the dose which is toxic. Indeed, the inventors have clearly shown that the lipid/alcohol vehicle can be combined with any NSAID to produce a supra-analgesic composition, in which high analgesic effects can be realised, while avoiding or at least reducing the risk that the patient will suffer the gut erosion side effect.

1-48. (canceled)

49. A pharmaceutical composition for oral administration, the composition comprising a therapeutically effective amount of a non-steroidal anti-inflammatory drug (NSAID) or a derivative thereof, and a pharmaceutically acceptable vehicle comprising a lipid and an alcohol, wherein the composition is for use in treating a respiratory disorder.

50. A composition according to claim 49, wherein the vehicle comprises at least about 50% (w/w), 60% (w/w), 70% (w/w), 80% (w/w) or at least about 90% (w/w) lipid.

51. A composition according to claim 49, wherein the vehicle comprises a lipid component selected from a group consisting of: an oil or oil-based liquid; a fat; a fatty acid (e.g. oleic acid, stearic acid or palmitic acid etc.), a fatty acid ester, a fatty alcohol, a glyceride (mono-, di- or tri-glyceride); a phospholipid; a glycol ester; a sucrose ester; a wax; a glycerol oleate derivative; a medium chain triglyceride; or a mixture thereof.

52. A composition according to claim 51, wherein the oil is a natural oil or a vegetable oil.

53. A composition according to claim 52, wherein the natural oil comprises linseed oil; soyabean oil; fractionated coconut oil; mineral oil; triacetin; ethyl oleate; a hydrogenated natural oil; or a mixture thereof.

54. A composition according to claim 52, wherein the vegetable oil is selected from a group consisting of olive oil; rapeseed oil; peanut oil; soybean oil; corn oil; safflower oil; arachis oil; sunflower oil; canola oil; walnut oil; almond oil; avocado oil; castor oil; coconut oil; corn oil; cottonseed oil; rice bran oil; sesame oil; and refined palm oil; or a mixture thereof.

55. A composition according to claim 49, wherein the lipid comprises a fatty acid selected from a group consisting of myristic acid (C 14:0); palmitic acid (C 16:0); palmitoleic acid (C 16:1); stearic acid (C 18:0); oleic acid (C 18:1); linoleic acid (C 18:2); linolenic acid (C 18:3) and arachidic acid (C 20:0); or a mixture thereof.

56. A composition according to claim 49, wherein the vehicle comprises less than about 50% (w/w) alcohol, preferably less than about 25% alcohol.

57. A composition according to claim 49, wherein the vehicle is selected from the following ranges: between approximately 80% and 95% (w/w) lipid, and between approximately 5% and 20% (w/w) ethanol or between 88% and 92% (w/w) lipid, and between approximately 8% and 12% (w/w) ethanol.

58. A composition according to claim 49, wherein the non-steroidal anti-inflammatory drug (NSAID) is a propionic acid derivative, an acetic acid derivative, an enolic acid derivative, a fenamic acid derivative, or a selective- or non-selective cyclooxygenase (COX) inhibitor.

59. A composition according to claim 49, wherein the non-steroidal anti-inflammatory drug is selected from a group consisting of: Alminoprofen; Benoxaprofen; Dexketoprofen; Flurbiprofen; Ibuprofen; Indoprofen; Ketoprofen; Loxoprofen; Pranoprofen; Protizinic acid; Suprofen; Aceclofenac; Acemetacin; Actarit; Alcofenac; Amfenac; Clometacin; Diclofenac; Etodolac; Felbinac; Fenclofenac; Indometacin; Ketorolac; Metiazinic acid; Mofezolac; Naproxen; Oxametacin; Sulindac; Zomepirac; Celecoxib; Etoricoxib; Lumiracoxib; Meloxicam; Rofecoxib; Valdecoxib; Aloxipirin; Aminophenazone; Antraphenine; Aspirin; Azapropazone; Benorilate; Benzydamine; Butibufen; Chlorthenoxacin; Choline Salicylate; Diflunisal; Emorfazone; Epirizole; Feclobuzone; Fenbufen; Glafenine; Hydroxylethyl salicylate; Lactyl phenetidin; Mefenamic acid; Metamizole; Mofebutazone; Nabumetone; Nifenazone; Niflumic acid; Phenacetin; Pipebuzone; Propyphenazone; Proquazone; Salicylamide; Salsalate; Tiaramide; Tinoridine; and Tolfenamic acid.

60. A composition according to claim 49, wherein the composition is for use in treating the common cold, sinusitis, tonsillitis, otitis media, pharyngitis, laryngitis, pneumonia, Respiratory Distress Syndrome (RDS), Acute Respiratory Distress Syndrome (ARDS), Acute Lung Injury (ALI), chronic obstructive pulmonary disorder (COPD), oedema, cystic fibrosis, a bacterial infection, a fungal infection, a viral infection or bronchiolitis.

61. A method of treating a respiratory disorder, the method comprising the step of administering an orally administrable pharmaceutical composition comprising a non-steroidal anti-inflammatory drug (NSAID) or a derivative thereof and a pharmaceutically acceptable vehicle comprising a lipid and an alcohol.

62. A method of treating inflammatory pain, the method comprising the step of administering an orally administrable pharmaceutical composition comprising a non-steroidal anti-inflammatory drug (NSAID) or a derivative thereof and a pharmaceutically acceptable vehicle comprising a lipid and an alcohol, wherein the NSAID or the derivative thereof is in a dose greater than 3200 mg/day.

63. A method of treating inflammatory pain, the method comprising the step of administering an over-the-counter (OTC), orally administrable pharmaceutical composition comprising a non-steroidal anti-inflammatory drug (NSAID) or a derivative thereof and a pharmaceutically acceptable vehicle comprising a lipid and an alcohol, wherein the NSAID or the derivative thereof is in a dose greater than 1600 mg/day.
